SEQUENCE LISTING (6 sequences)

SEQ ID NO: 1 - protein, 233 aa, Artificial Sequence
  derivative of the full length ORF2695e
EDSKETEIKQNFNKMLNVYPTKNLEDFYDKEGFRDEEFDKGDKGTWIIRSEMTKQPKGKIMTSRGMVLYINRNTRTAKGYFLIDEIKDDNSGRPIENEKKYPVKMNHNKIFPTKPISDDKLKKEIENFKFFVQYGDFKNLKDYKDGEISYNPNVPSYSAQYQLNNNDNNVKQLRKRYDIPTNQAPKLLLKGDGDLKGSSVGSKNLEFTFVENKEENIFFTDAVQFTPSEDDES

SEQ ID NO: 2 - protein, 257 aa, Artificial Sequence
  His-Tag derivative of SEQ ID NO 1
MGHHHHHHHHHHSSGHIEGRHMLEEDSKETEIKQNFNKMLNVYPTKNLEDFYDKEGFRDEEFDKGDKGTWIIRSEMTKQPKGKIMTSRGMVLYINRNTRTAKGYFLIDEIKDDNSGRPIENEKKYPVKMNHNKIFPTKPISDDKLKKEIENFKFFVQYGDFKNLKDYKDGEISYNPNVPSYSAQYQLNNNDNNVKQLRKRYDIPTNQAPKLLLKGDGDLKGSSVGSKNLEFTFVENKEENIFFTDAVQFTPSEDDES

SEQ ID NO: 3 - protein, 261 aa, Staphylococcus epidermidis
MRYLKKVTIYISLLILTIFIGGCGFINKEDSKETEIKQNFNKMLNVYPTKNLEDFYDKEGFRDEEFDKGDKGTWIIRSEMTKQPKGKIMTSRGMVLYINRNTRTAKGYFLIDEIKDDNSGRPIENEKKYPVKMNHNKIFPTKPISDDKLKKEIENFKFFVQYGDFKNLKDYKDGEISYNPNVPSYSAQYQLNNNDNNVKQLRKRYDIPTNQAPKLLLKGDGDLKGSSVGSKNLEFTFVENKEENIFFTDAVQFTPSEDDES

SEQ ID NO: 4 - DNA, 774 bp, Staphylococcus epidermidis
atgggccatcatcatcatcatcatcatcatcatcacagcagcggccatatcgaaggtcgtcatatgctcgaggaagatagcaaagaaacggaaatcaaacaaaactttaataagatgttaaacgtgtatcctactaaaaatctagaagacttttatgataaagaaggttttcgagatgaagaatttgataaaggagataaaggaacttggattattaggtctgaaatgacaaaacagccaaaaggtaaaattatgacctcaagaggtatggttctctatatcaatcgtaacactagaacagccaaagggtattttttaatagatgagataaaagatgataatagtggtagaccgatagagaatgaaaagaaataccctgtaaaaatgaaccataataagatctttccaacaaagccaatatctgatgataaattaaaaaaagaaattgaaaacttcaaattttttgtacaatatggagattttaaaaacttaaaggattataaagacggagagatatcttacaatcctaacgttcctagttactcagcgcaatatcaattgaacaataatgataataatgttaaacaattaagaaaaagatatgatattccaaccaatcaagcccctaaattattgttaaaaggggatggcgacttaaaaggatcatctgtaggttctaaaaatttagaatttacttttgtagaaaataaagaagagaatatattttttacagatgcagtacaattcactcctagcgaggatgatgaatcatga

SEQ ID NO: 5 - DNA, 38 bp, Artificial Sequence
  PCR primer
ggaattcgctcagctcatgattcatcatcctcgctagg

SEQ ID NO: 6 - DNA, 35 bp, Artificial Sequence
  PCR primer
ggatatcctcgaggaagatagcaaagaaacggaaa

(19) United States
(12) Patent Application Publication
(10) Pub. No.: US 2010/0104591 A1
(43) Pub. Date: Apr. 29, 2010

(54) POLYPEPTIDES FOR INDUCING A PROTECTIVE IMMUNE RESPONSE AGAINST STAPHYLOCOCCUS EPIDERMIDIS

(76) Inventors: Annaliesa S. Anderson, Upper Saddle River, NJ (US); Tessie McNeely, Gwynedd Valley, PA (US); James C. Cook III, North Wales, PA (US); William L. McClements, Doylestown, PA (US); Donna L. Montgomery, Chalfont, PA (US)

Correspondence Address: MERCK, P O BOX 2000, RAHWAY, NJ, 07065-0907, US

(21) Appl. No.: 12/531,107

(22) PCT Filed: Mar. 14, 2008
(86) PCT No.: PCT/US08/03389

§ 371 Date: Sep. 14, 2009

Related U.S. Application Data
(60) Provisional application No. 60/918,846, filed on Mar. 19, 2007.

(51) Int. Cl. A61K 39/00; C07K 14/00; C07H 21/04; A61P 37/04; A61P 31/04; C12N 15/63; C12P 21/00

(52) U.S. Cl. 424/185.1; 530/350; 536/23.1; 435/320.1; 435/69.3

(57) ABSTRACT

The present invention features polypeptides comprising an amino acid sequence structurally related to SEQ ID NO: 1 and uses of such polypeptides. SEQ ID NO: 1 is a truncated derivative of a full-length S. epidermidis polypeptide. The full-length naturally occurring polypeptide is referred to herein as full-length ORF2695e. A His-tagged derivative of SEQ ID NO: 1 was found to produce a protective immune response against S. epidermidis.

### CROSS-REFERENCE TO RELATED APPLICATIONS

This application claims the benefit of U.S. Provisional Application No. 60/918,846 filed Mar. 19, 2007, herein incorporated by reference.

### BACKGROUND OF THE INVENTION

The references cited throughout the present application are not admitted to be prior art to the claimed invention.

Staphylococcus epidermidis has emerged as pathogen, particularly in nosocomial and immune compromised patients. (Ziebuhr et al., International Journal of Antimicrobial Agents 28S:S14-S20, 2006.) Coagulase-negative staphylococci (CoNS), mainly S. epidermidis, are the most frequently isolated microorganism infection associated with foreign bodies used in diagnostic or therapeutic procedures. (Heilmann and Peters, Biology and Pathogenicity of Staphylococcus epidermidis, In: Gram Positive Pathogens, Eds. Fischetti et al., American Society for Microbiology, Washington D.C. 2000 and The Staphylococci in Human Disease, Crossley and Archer (eds.), Churchill Livingstone Inc. 1997.)

Nucleic acid from S. epidermis has been sequenced to obtain nucleic acid sequence information and make predictions concerning open reading frames and potential polypeptides. (Doucette-Stamm et al., U.S. Pat. No. 6,380,370 and Doucette-Stamm et al., U.S. Pat. No. 7,060,458.)

Techniques such as those involving display technology and sera from infected patients can be used in an effort to identify genes coding for potential antigens. (Meinke et al.,

International Publication Number WO 02/059148, Meinke et al., International Publication Number WO 04/087746.)

### SUMMARY OF THE INVENTION

The present invention features polypeptides comprising an amino acid sequence structurally related to SEQ ID NO: 1 and uses of such polypeptides. SEQ ID NO: 1 is a truncated derivative of a full-length S. epidermidis polypeptide. The full-length naturally occurring polypeptide is referred to herein as full-length ORF2695e. A His-tagged derivative of SEQ ID NO: 1 was found to produce a protective immune response against S. epidermidis.

Reference to “protective” immunity or immune response indicates a detectable level of protection against S. epidermidis infection. Reference to “immunogen” indicates the ability to provide protective immunity.

Thus, a first aspect of the present invention describes a polypeptide immunogen comprising an amino acid sequence at least 90% identical to SEQ ID NO: 1, wherein the polypeptide does not have the amino acid sequence of SEQ ID NO: 3. Reference to comprising an amino acid sequence at least 90% identical to SEQ ID NO: 1 indicates that a SEQ ID NO: 1 related region is present and additional regions may be present. In an embodiment, if additional regions are present, the polypeptide does not have an amino terminus provided by amino acids 1-28 of SEQ ID NO: 3.

Percent identity (also referred to as percent identical) to a reference sequence is determined by aligning the polypeptide sequence with the reference sequence and determining the number of identical amino acids in the corresponding regions. This number is divided by the total number of amino acids in the reference sequence (e.g., SEQ ID NO: 1) and then multiplied by 100 and rounded to the nearest whole number.

Another aspect of the present invention describes an immunogen comprising an amino acid sequence that provides protective immunity against S. epidermidis and one or more additional regions or moieties covalently joined to the amino acid sequence at the carboxyl terminus or amino terminus, wherein each region or moiety is independently selected from a region or moiety having at least one of the following properties: enhances the immune response, facilitates purification, or facilitates polypeptide stability.

Reference to “additional region or moiety” indicates a region or moiety different from a ORF2695e region. The additional region or moiety can be, for example, an additional polypeptide region or a non-peptide region.

Another aspect of the present invention describes a composition able to induce protective immunity against S. epidermidis in a patient. The composition comprises a pharmaceutically acceptable carrier and an immunologically effective amount of an immunogen that provides protective immunity against S. epidermidis.

An immunologically effective amount is an amount sufficient to provide protective immunity against S. epidermidis infection. The amount should be sufficient to significantly prevent the likelihood or severity of a S. epidermidis infection.

Another aspect of the present invention describes a nucleic acid comprising a recombinant gene encoding a polypeptide that provides protective immunity against S. epidermidis. A recombinant gene contains recombinant nucleic acid encoding a polypeptide along with regulatory elements for proper transcription and processing (which may include translational and post translational elements). The recombinant gene can exist independent of a host genome or can be part of a host genome.

A recombinant nucleic acid is nucleic acid that by virtue of its sequence and/or form does not occur in nature. Examples of recombinant nucleic acid include purified nucleic acid, two or more nucleic acid regions combined together that provides a different nucleic acid than found in nature, and the absence of one or more nucleic acid regions (e.g., upstream or downstream regions) that are naturally associated with each other.

Another aspect of the present invention describes a recombinant cell. The cell comprises a recombinant gene encoding a polypeptide that provides protective immunity against S. epidermidis. Preferably, the cell is grown in vitro.

Another aspect of the present invention describes a method of making a polypeptide that provides protective immunity against S. epidermidis. The method involves growing a recombinant cell containing recombinant nucleic acid encoding the polypeptide and purifying the polypeptide.

Another aspect of the present invention describes a polypeptide that provides protective immunity against S. epidermidis made by a process comprising the steps of growing a recombinant cell containing recombinant nucleic acid encoding the polypeptide in a host and purifying the polypeptide. Different host cells can be employed.

Another aspect of the present invention describes a method of inducing a protective immune response in a patient against S. epidermidis. The method comprises the step of administering to the patient an immunologically effective amount of an immunogen providing protective immunity against S. epidermidis.

Unless particular terms are mutually exclusive, reference to “or” indicates either or both possibilities. Occasionally phrases such as “and/or” are used to highlight either or both possibilities.

Reference to open-ended terms such as “comprises” allows for additional elements or steps. Occasionally phrases such as “one or more” are used with or without open-ended terms to highlight the possibility of additional elements or steps.

Unless explicitly stated reference to terms such as “a” or “an” is not limited to one. For example, “a cell” does not exclude “cells”. Occasionally phrases such as one or more are used to highlight the possible presence of a plurality.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 illustrates the amino acid sequence of SEQ ID NO: 2. SEQ ID NO: 2 is a His-Tag derivative of SEQ ID NO: 1. The SEQ ID NO: 1 region is shown in bold.

FIGS. 2A and 2B illustrate the full-length ORF2695e of SEQ ID NO: 3 (FIG. 2A) and an encoding nucleic acid (FIG. 2B). The SEQ ID NO: 1 region is shown in bold in FIG. 2A. The SEQ ID NO: 1 encoding region is shown in bold in FIG. 2B.

### DETAILED DESCRIPTION OF THE INVENTION

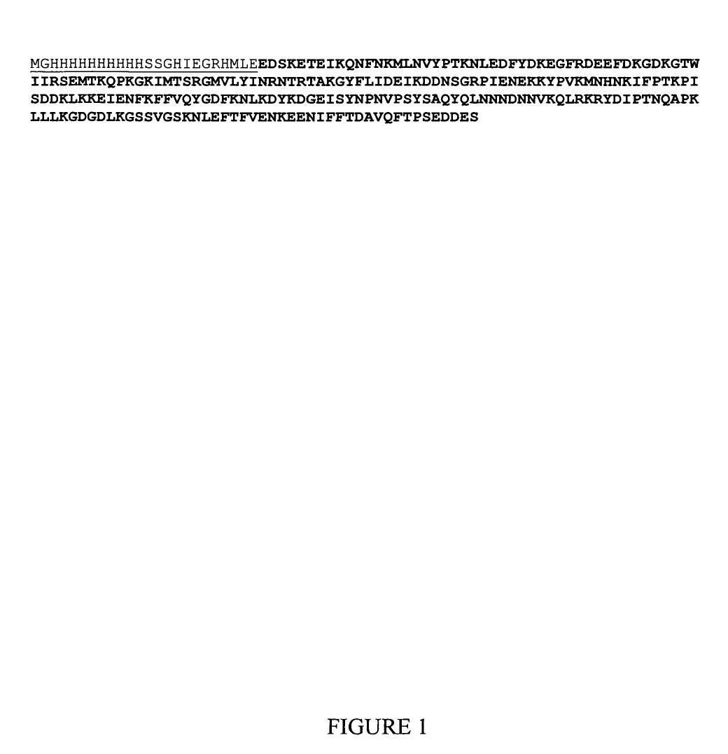

The ability of SEQ ID NO: 1 related polypeptides to provide protective immunity is illustrated in the Examples provided below using SEQ ID NO: 2. SEQ ID NO: 2 is a His-Tag derivative of SEQ ID NO: 1. The His-tag facilitates polypeptide purification and identification. FIG. 1 illustrates SEQ ID NO: 2, where the SEQ ID NO: 1 region is shown in bold.

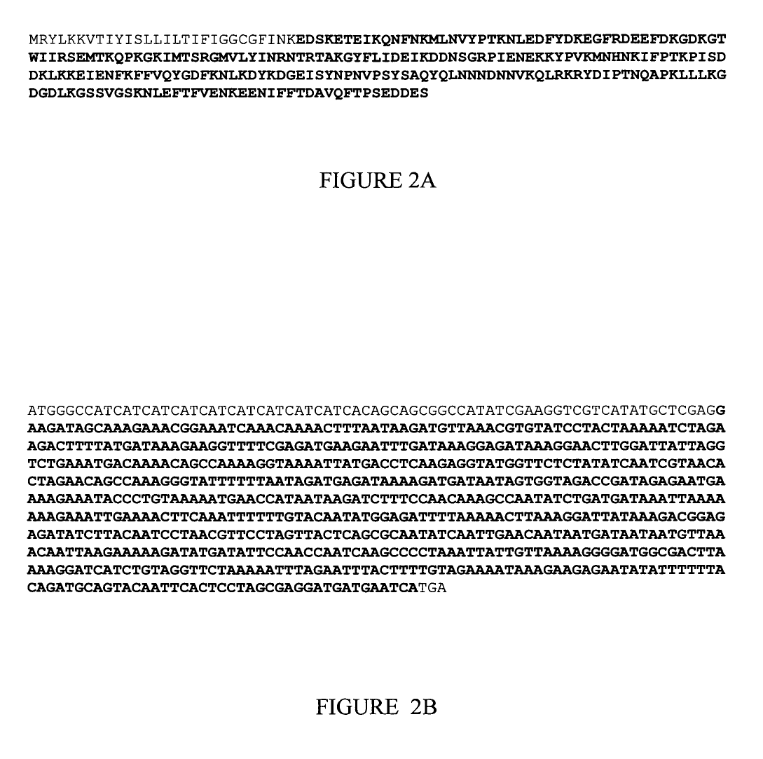

SEQ ID NO: 1 is a derivative of the full length ORF2695e S. epidermidis polypeptide. SEQ ID NO: 1 contains amino acids 29-261 of a ORF2695e sequence (SEQ ID NO: 3). Amino acids 1-28 of SEQ ID NO: 3 were identified as a leader sequence. FIGS. 2A and 2B illustrate SEQ ID NO: 3 and an encoding nucleic acid sequence, where the SEQ ID NO: 1 region is shown in bold.

### ORF2695e Sequences

ORF2695e has an amino acid sequence corresponding to Gen-Bank Accession No. Q5HKC6. Gen-Bank Accession No. Q5HKC6 references Gill et al., J. Bacteria 187(7):2426-2438, 2005.

Other naturally occurring ORF2695e sequences can be identified based on the presence of a high degree of sequence similarity or contiguous amino acids compared to a known ORF2695e sequence. Contiguous amino acids provide characteristic tags. In different embodiments, a naturally occurring ORF2695e sequence is a sequence found in a Staphylococcus sp, preferably S. epidermidis, having at least 20, at least 30, or at least 50 contiguous amino acids as in SEQ ID NO: 1; and/or having at least 90% sequence similarity or identity with SEQ ID NO: 1.

Sequence similarity can be determined by different algorithms and techniques well known in the art. Generally, sequence similarity is determined by techniques aligning two sequences to obtain maximum amino acid identity, allowing for gaps, additions and substitutions in one of the sequences.

Sequence similarity can be determined, for example, using a local alignment tool utilizing the program align (developed by Huang and Miller, Adv. Appl. Math. 12:337-357, 1991, for the <<sim>> program). The options and environment variables are:-f# Penalty for the first residue a gap (−14 by default); −g # Penalty for each additional residue in a gap (−4 by default)-s str (SMATRIX) the filename of an alternative scoring matrix file. For protein sequences, PAM250 is used by default-w # (LINLEN) output line length for sequence alignments (60).

### SEQ ID NO: 1 Related Polypeptides

Polypeptides structurally related to SEQ ID NO: 1 include polypeptides containing corresponding regions present in different S. epidermidis strains and derivatives of naturally occurring regions. SEQ ID NO: 1 related polypeptides contain an amino acid sequence at least 90% identical to SEQ ID NO: 1. Reference to “polypeptide” does not provide a minimum or maximum size limitation.

A polypeptide at least 90% identical to SEQ ID NO: 1 contains up to about 26 amino acid alterations from SEQ ID NO: 1. Each amino acid alteration is independently an amino acid substitution, deletion, or addition. The alterations can be within the SEQ ID NO: 1 region or added to the SEQ ID NO: 1 region. In different embodiments, the SEQ ID NO: 1 related polypeptide is at least 94%, or at least 99% identical to SEQ ID NO: 1; differs from SEQ ID NO: 1 by 0, 1, 2, 3, 4, 5, 6, 7, 8, 9, 10, 11, 12, 13, 14, 15, 16, 17, 18, 19, or 20 amino acid alterations; or consists essentially of SEQ ID NO: 1.

Reference to “consists essentially” of indicated amino acids indicates that the referred to amino acids are present and additional amino acids may be present. The additional amino acids can be at the carboxyl or amino terminus. In different embodiments 1, 2, 3, 4, 5, 6, 7, 8, 9, 10, 11, 12, 13, 14, 15, 16, 17, 18, 19, or 20 additional amino acids are present. A preferred additional amino acid is an amino terminus methionine.

Alterations can be made to SEQ ID NO: 1 to obtain derivatives that can induce protective immunity against S. epidermidis. Alterations can be performed, for example, to obtain a derivative retaining the ability to induce protective immunity against S. epidermidis or to obtain a derivative that in addition to providing protective immunity also has a region that can achieve a particular purpose.

Alterations can be made taking into account different ORF2695e sequences and known properties of amino acids. Generally, in substituting different amino acids to retain activity it is preferable to exchange amino acids having similar properties. Factors that can be taken into account for an amino acid substitution include amino acid size, charge, polarity, and hydrophobicity. The effect of different amino acid R-groups on amino acid properties are well known in the art. (See, for example, Ausubel, Current Protocols in Molecular Biology, John Wiley, 1987-2002, Appendix 1C.)

Alterations to achieve a particular purpose include those designed to facilitate production or efficacy of the polypeptide; or cloning of the encoded nucleic acid. Polypeptide production can be facilitated through the use of an initiation codon (e.g., coding for methionine) suitable for recombinant expression. The methionine may be later removed during cellular processing. Cloning can be facilitated by, for example, the introduction of restriction sites which can be accompanied by amino acid additions or changes.

Efficacy of a polypeptide to induce an immune response can be enhanced through epitope enhancement. Epitope enhancement can be performed using different techniques such as those involving alteration of anchor residues to improve peptide affinity for MHC molecules and those increasing affinity of the peptide-MHC complex for a T-cell receptor. (Berzofsky et al., Nature Review 1:209-219, 2001.)

Preferably, the polypeptide is a purified polypeptide. A “purified polypeptide” is present in an environment lacking one or more other polypeptides with which it is naturally associated and/or is represented by at least about 10% of the total protein present. In different embodiments, the purified polypeptide represents at least about 50%, at least about 75%, or at least about 95% of the total protein in a sample or preparation.

In an embodiment, the polypeptide is “substantially purified.” A substantially purified polypeptide is present in an environment lacking all, or most, other polypeptides with which the polypeptide is naturally associated. For example, a substantially purified S. epidermidis polypeptide is present in an environment lacking all, or most, other S. epidermidis polypeptides. An environment can be, for example, a sample or preparation.

Reference to “purified” or “substantially purified” does not require a polypeptide to undergo any purification and may include, for example, a chemically synthesized polypeptide that has not been purified.

Polypeptide stability can be enhanced by modifying the polypeptide carboxyl or amino terminus. Examples of possible modifications include amino terminus protecting groups such as acetyl, propyl, succinyl, benzyl, benzyloxycarbonyl or t-butyloxycarbonyl; and carboxyl terminus protecting groups such as amide, methylamide, and ethylamide.

In an embodiment, the polypeptide immunogen is part of an immunogen containing one or more additional regions or moieties covalently joined to the polypeptide at the carboxyl terminus or amino terminus, where each region or moiety is independently selected from a region or moiety having at least one of the following properties: enhances the immune response, facilitates purification, or facilitates polypeptide stability. Polypeptide stability can be enhanced, for example, using groups such as polyethylene glycol that may be present on the amino or carboxyl terminus.

Polypeptide purification can be enhanced by adding a group to the carboxyl or amino terminus to facilitate purification. Examples of groups that can be used to facilitate purification include polypeptides providing affinity tags. Examples of affinity tags include a six-histidine tag, trpE, glutathione and maltose-binding protein.

The ability of a polypeptide to produce an immune response can be enhanced using groups that generally enhance an immune response. Examples of groups that can be joined to a polypeptide to enhance an immune response against the polypeptide include cytokines such as IL-2. (Buchan et al., 2000. Molecular Immunology 37:545-552.)

### Polypeptide Production

Polypeptides can be produced using standard techniques including those involving chemical synthesis and those involving purification from a cell producing the polypeptide. Techniques for chemical synthesis of polypeptides are well known in the art. (See e.g., Vincent, Peptide and Protein Drug Delivery, New York, N.Y., Decker, 1990.) Techniques for recombinant polypeptide production and purification are also well known in the art. (See for example, Ausubel, Current Protocols in Molecular Biology, John Wiley, 1987-2002.)

Obtaining polypeptides from a cell is facilitated using recombinant nucleic acid techniques to produce the polypeptide. Recombinant nucleic acid techniques for producing a polypeptide involve introducing, or producing, a recombinant gene encoding the polypeptide in a cell and expressing the polypeptide.

A recombinant gene contains nucleic acid encoding a polypeptide along with regulatory elements for polypeptide expression. The recombinant gene can be present in a cellular genome or can be part of an expression vector.

The regulatory elements that may be present as part of a recombinant gene include those naturally associated with the polypeptide encoding sequence and exogenous regulatory elements not naturally associated with the polypeptide encoding sequence. Exogenous regulatory elements such as an exogenous promoter can be useful for expressing a recombinant gene in a particular host or increasing the level of expression. Generally, the regulatory elements that are present in a recombinant gene include a transcriptional promoter, a ribosome binding site, a terminator, and an optionally present operator. A preferred element for processing in eukaryotic cells is a polyadenylation signal.

Expression of a recombinant gene in a cell is facilitated through the use of an expression vector. Preferably, an expression vector in addition to a recombinant gene also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

Due to the degeneracy of the genetic code, a large number of different encoding nucleic acid sequences can be used to code for a particular polypeptide. The degeneracy of the genetic code arises because almost all amino acids are encoded by different combinations of nucleotide triplets or “codons”. Amino acids are encoded by codons as follows:
A=Ala—Alanine: codons GCA, GCC, GCG, GCU
C=Cys—Cysteine: codons UGC, UGU
D=Asp—Aspartic acid: codons GAC, GAU
E=Glu—Glutamic acid: codons GAA, GAG
F=Phe—Phenylalanine: codons UUC, UUU
G=Gly—Glycine: codons GGA, GGC, GGG, GGU
H=His—Histidine: codons CAC, CAU
I=Ile—Isoleucine: codons AUA, AUC, AUU
K=Lys—Lysine: codons AAA, AAG
L=Leu—Leucine: codons UUA, UUG, CUA, CUC, CUG, CUU
M=Met—Methionine: codon AUG
N=Asn—Asparagine: codons AAC, AAU
P=Pro—Proline: codons CCA, CCC, CCG, CCU
Q=Gln—Glutamine: codons CAA, CAG
R=Arg—Arginine: codons AGA, AGG, CGA, CGC, CGG, CGU
S=Ser—Serine: codons AGC, AGU, UCA, UCC, UCG, UCU
T=Thr—Threonine: codons ACA, ACC, ACG, ACU
V=Val—Valine: codons GUA, GUC, GUG, GUU
W=Trp—Tryptophan: codon UGG
Y=Tyr—Tyrosine: codons UAC, UAU

Suitable cells for recombinant nucleic acid expression of SEQ ID NO: 1 related polypeptides are prokaryotes and eukaryotes. Examples of prokaryotic cells include E. coli; members of the Staphylococcus genus, such as S. epidermidis; members of the Lactobacillus genus, such as L. plantarum; members of the Lactococcus genus, such as L. lactis; members of the Bacillus genus, such as B. subtilis; members of the Corynebacterium genus such as C. glutamicum; and members of the pseudomonas genus such as Ps. fluorescens. Examples of eukaryotic cells include mammalian cells; insect cells; yeast cells such as members of the Saccharomyces genus (e.g., S. cerevisiae), members of the Pichia genus (e.g., P. pastoris), members of the Hansenula genus (e.g., H. polymorpha), members of the Kluyveromyces genus (e.g., K. lactis or K. fragilis) and members of the Schizosaccharomyces genus (e.g., S. pombe).

Techniques for recombinant gene production, introduction into a cell, and recombinant gene expression are well known in the art. Examples of such techniques are provided in references such as Ausubel, Current Protocols in Molecular Biology, John Wiley, 1987-2002, and Sambrook et al., Molecular Cloning, A Laboratory Manual, 2^{nd} Edition, Cold

Spring Harbor Laboratory Press, 1989.

If desired, expression in a particular host can be enhanced through codon optimization. Codon optimization includes use of more preferred codons. Techniques for codon optimization in different hosts are well known in the art. SEQ ID NO: 1 related polypeptides may contain post translational modifications, for example, N-linked glycosylation, O-linked glycosylation, or acetylation. Reference to “polypeptide” or an “amino acid” sequence of a polypeptide includes polypeptides containing one or more amino acids having a structure of a post-translational modification from a host cell, such as a yeast host.

Post translational modifications can be produced chemically or by making use of suitable hosts. For example, in S. cerevisiae the nature of the penultimate amino acid appears to determine whether the N-terminal methionine is removed. Furthermore, the nature of the penultimate amino acid also determines whether the N-terminal amino acid is N^{α}-acetylated (Huang et al., Biochemistry 26: 8242-8246, 1987). Another example includes a polypeptide targeted for secretion due to the presence of a secretory leader (e.g., signal peptide), where the protein is modified by N-linked or O-linked glycosylation. (Kukuruzinska et al., Ann. Rev. Biochem. 56:915-944, 1987.)

### Adjuvants

Adjuvants are substances that can assist an immunogen in producing an immune response. Adjuvants can function by different mechanisms such as one or more of the following: increasing the antigen biologic or immunologic half-life; improving antigen delivery to antigen-presenting cells; improving antigen processing and presentation by antigen-presenting cells; and inducing production of immunomodulatory cytokines. (Vogel, Clinical Infectious Diseases 30(suppl. 3):S266-270, 2000.) In an embodiment, an adjuvant is used.

A variety of different types of adjuvants can be employed to assist in the production of an immune response. Examples of particular adjuvants include aluminum hydroxide, aluminum phosphate, or other salts of aluminum, calcium phosphate, DNA CpG motifs, monophosphoryl lipid A, cholera toxin, E. coli heat-labile toxin, pertussis toxin, muramyl dipeptide, Freund's incomplete adjuvant, MF59, SAF, immunostimulatory complexes, liposomes, biodegradable microspheres, saponins, nonionic block copolymers, muramyl peptide analogues, polyphosphazene, synthetic polynucleotides, IFN-γ, IL-2, IL-12, and ISCOMS. (Vogel Clinical Infectious Diseases 30(suppl 3):S266-270, 2000, Klein et al., Journal of Pharmaceutical Sciences 89:311-321, 2000, Rimmelzwaan et al., Vaccine 19:1180-1187, 2001, Kersten Vaccine 21:915-920, 2003, O'Hagen Curr. Drug Target Infect. Disord., 1:273-286, 2001.)

### Patients For Inducing Protective Immunity

A “patient” refers to a mammal capable of being infected with S. epidermidis. A patient can be treated prophylactically or therapeutically. Prophylactic treatment provides sufficient protective immunity to reduce the likelihood, or severity, of a S. epidermidis infection. Therapeutic treatment can be performed to reduce the severity of a S. epidermidis infection.

Prophylactic treatment can be performed using a vaccine containing an immunogen described herein. Such treatment is preferably performed on a human. Vaccines can be administered to the general population or to those persons at an increased risk of S. epidermidis infection.

Persons with an increased risk of S. epidermidis infection include health care workers; hospital patients; patients with a weakened immune system; patients undergoing surgery; patients receiving foreign body implants, such a catheter or a vascular device; patients facing therapy leading to a weakened immunity; patients under diagnostic procedures involving foreign bodies; and persons in professions having an increased risk of burn or wound injury.

Foreign bodies used in diagnostic or therapeutic procedures include indwelling catheters or implanted polymer device. Examples of foreign bodies associated S. epidermidis infections include stepticemia/endocarditis (e.g., intravascular catheters, vascular prostheses, pacemaker leads, defibrillator systems, prosthetic heart valves, and left ventricular assist devices); peritonitis (e.g., ventriculo-peritoneal cerebrospinal fluid (CSF) shunts and continuous ambulatory peritoneal dialysis catheter systems); ventriculitis (e.g., internal and external CSF shunts); and chronic polymer-associated syndromes (e.g., prosthetic joint (hip) loosening, fibrous capsular contracture syndrome after mammary argumentation with silicone prosthesis and late-onset endophtalmisis after implantation of artificial intraocular lenses following cataract surgery). (Heilmann and Peters, Biology and Pathogenicity of Staphylococcus epidermidis, In: Gram Positive Pathogens, Eds. Fischetti et al., American Society for Microbiology, Washington D.C. 2000.)

Non-human patients that can be infected with S. epidermidis include cows, pigs, sheep, goats, rabbits, horses, dogs, cats and mice. Treatment of non-human patients is useful in protecting pets and livestock, and in evaluating the efficacy of a particular treatment.

In an embodiment, a patient is treated prophylactically in conjunction with a therapeutic or medical procedure involving a foreign body. In additional embodiments, the patient is immunized at about 1 month, about 2 month or about 2-6 months prior to the procedure.

### Combination Vaccines

SEQ ID NO: 1 related polypeptides can be used alone, or in combination with other immunogens, to induce an immune response. Additional immunogens that may be present include one or more additional S. epidermidis immunogens, one or more immunogens targeting one or more other Staphylococcus organisms such as S. aureus, S. haemolyticus, S. warneri, or S. lugunensi, and/or one or more immunogens targeting other infections organisms.

Examples of one or more additional immunogens include ORF0657n related polypeptides (Anderson et al., International Publication No. WO 05/009379); ORF0657/ORF0190 hybrid polypeptides (Anderson et al., International Publication No. WO 05/009378); sai-1 related polypeptides (Anderson et al., International Publication No. WO 05/79315); ORF0594 related polypeptides (Anderson et al., International Publication No. WO 05/086663); ORF0826 related polypeptides (Anderson et al., International Publication No. WO 05/115113); PBP4 related polypeptides (Anderson et al., International Publication No. WO 06/033918); AhpC related polypeptides and AhpC-AhpF compositions (Kelly et al. International Publication No. WO 06/078680); S. aureus type 5 and type 8 capsular polysaccharides (Shinefield et al., N. Eng. J. Med. 346:491-496, 2002); collagen adhesin, fibrinogen binding proteins, and clumping factor (Mamo et al., FEMS Immunology and Medical Microbiology 10:47-54, 1994, Nilsson et al., J. Clin. Invest. 101:2640-2649, 1998, Josefsson et al., The Journal of Infectious Diseases 184:1572-1580, 2001) and polysaccharide intercellular adhesin and fragments thereof (Joyce et al., Carbohydrate Research 338:903-922, 2003).

### Administration

Immunogens can be formulated and administered to a patient using the guidance provided herein along with techniques well known in the art. Guidelines for pharmaceutical administration in general are provided in, for example, Vaccines Eds. Plotkin and Orenstein, W. B. Sanders Company, 1999; Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990.

Pharmaceutically acceptable carriers facilitate storage and administration of an immunogen to a patient. Pharmaceutically acceptable carriers may contain different components such as a buffer, sterile water for injection, normal saline or phosphate buffered saline, sucrose, histidine, salts and polysorbate.

Immunogens can be administered by different routes such as subcutaneous, intramuscular, or mucosal. Subcutaneous and intramuscular administration can be performed using, for example, needles or jet-injectors.

Suitable dosing regimens are preferably determined taking into account factors well known in the art including age, weight, sex and medical condition of the patient; the route of administration; the desired effect; and the particular compound employed. The immunogen can be used in multi-dose vaccine formats. It is expected that a dose would consist of the range of 1.0 μg to 1.0 mg total polypeptide. In different embodiments of the present invention the range is from 5.0 μg to 500 μg, 0.01 mg to 1.0 mg or 0.1 mg to 1.0 mg.

The timing of doses depends upon factors well known in the art. After the initial administration one or more booster doses may subsequently be administered to maintain or boost antibody titers. An example of a dosing regime would be day 1, 1 month, a third dose at either 4, 6 or 12 months, and additional booster doses at distant times as needed.

### Generation of Antibodies

A SEQ ID NO: 1 related polypeptide can be used to generate antibodies and antibody fragments binding to the polypeptide or to S. epidermidis. Such antibodies and antibody fragments have different uses including use in polypeptide purification, S. epidermidis identification, or in therapeutic or prophylactic treatment against S. epidermidis infection.

Antibodies can be polyclonal or monoclonal. Techniques for producing and using antibodies, including human antibodies, are well known in the art. (Ausubel, Current Protocols in Molecular Biology, John Wiley, 1987-2002, Harlow et al., Antibodies, A Laboratory Manual, Cold Spring Harbor Laboratory, 1988, Kohler et al., Nature 256:495-497, 1975, Azzazy et al., Clinical Biochemistry 35:425-445, 2002, Berger et al., Am. J. Med. Sci. 324(1):14-40, 2002.)

Proper glycosylation can be important for antibody function. (Yoo et al., Journal of Immunological Methods 261:1-20, 2002, Li et al., Nature Biotechnology 24(2):210-215, 2006.) Naturally occurring antibodies contain at least one N-linked carbohydrate attached to a heavy chain. (Yoo et al., Journal of Immunological Methods 261:1-20, 2002.) Additional N-linked carbohydrates and O-linked carbohydrates may be present and may be important for antibody function. (Id.)

Different types of host cells can be used to provide for efficient post-translational modifications including mammalian host cells and non-mammalian cells. Examples of mammalian host cells include Chinese hamster ovary (Cho), HeLa, C6, PC12, and myeloma cells. (Yoo et al., Journal of Immunological Methods 261:1-20, 2002, Persic et al., Gene 187:9-18, 1997.) Non-mammalian cells can be modified to replicate human glycosylation. (Li et al., Nature Biotechnology 24(2):210-215, 2006.) Glycoenginnered Pichia pastoris is an example of such a modified non-mammalian cell. (Li et al., Nature Biotechnology 24(2):210-215, 2006.)

### Nucleic Acid Vaccine

Nucleic acid encoding a SEQ ID NO: 1 related polypeptide can be introduced into a patient using vectors suitable for therapeutic administration. Suitable vectors can deliver nucleic acid into a target cell without causing an unacceptable side effect. Examples of vectors that can be employed include plasmid vectors and viral based vectors. (Barouch J. Pathol. 208:283-289, 2006, Emini et al., International Publication No. WO 03/031588.)

Cellular expression is achieved using a gene expression cassette encoding a desired polypeptide. The gene expression cassette contains regulatory elements for producing and processing a sufficient amount of nucleic acid inside a target cell to achieve a beneficial effect.

Examples of viral vectors include first and second generation adenovectors, helper dependent adenovectors, adeno-associated viral vectors, retroviral vectors, alpha virus vectors, Venezuelan Equine Encephalitis virus vector, and plasmid vectors. (Hitt et al., Advances in Pharmacology 40:137-206, 1997, Johnston et al., U.S. Pat. No. 6,156,588, Johnston et al., International Publication No. WO 95/32733, Barouch J. Pathol. 208:283-289, 2006, Emini et al., International Publication No. WO 03/031588.)

Adenovectors can be based on different adenovirus serotypes such as those found in humans or animals. Examples of animal adenoviruses include bovine, porcine, chimpanzee, murine, canine, and avian (CELO). (Emini et al., International Publication No. WO 03/031588, Colloca et al., International Publication No. WO 05/071093.) Human adenovirus include Group B, C, D, or E serotypes such as type 2 (“Ad2”), 4 (“Ad4”), 5 (“Ad5”), 6 (“Ad6”), 24 (“Ad24”), 26 (“Ad26”), 34 (“Ad34”) and 35 (“Ad35”).

Nucleic acid vaccines can be administered using different techniques and dosing regimes. (Emini et al., International Publication No. WO 03/031588.) For example, the vaccine can be administered intramuscular by injection with or without one or more electric pulses. Electric mediated transfer can assist genetic immunization by stimulating both humoral and cellular immune responses. Examples of dosing regimes include prime-boost and heterologous prime-boost approaches. (Emini et al., International Publication No. WO 03/031588.)

### EXAMPLES

Examples are provided below further illustrating different features of the present invention. The examples also illustrate useful methodology for practicing the invention. These examples do not limit the claimed invention.

### Example 1

### Protective Immunogen Production, Purification, and Formulation

This example describes SEQ ID NO: 2 production, purification, and formulation. SEQ ID NO: 2 was used in the examples described below to illustrate the ability of SEQ ID NO: 1 related polypeptides to provide protective immunity. SEQ ID NO: 2 is a His-tagged derivative of SEQ ID NO: 1.

### ORF2695e Cloning and Expression and Modification

The complete open reading frame for ORF2695e was analysed using SignalP, a program designed to identify putative signal sequences. A potential cleavage site was detected after aa 28. PCR primers were therefore designed to amplify nucleotides that would code from aa 29 to the end of the protein. Restriction sites were also added to the PCR primers to facilitate cloning into the pET-16b vector. The restriction sites were: a XhoI site on the carboxy terminal end and a BpuI site on the amino termini (Table 1). The final expressed construct was designed to have a HIS-tag on the amino terminal end to facilitate purification.

TABLE 1

| | Sequence (non | |
|---|---|---|
| | ORF2695e | |
| Primer | sequence underlined) | |
| Primer 1 | GGAATTC GCTCAGC TCA | Amino Primer |
| SEQ ID NO: 5 | TGATTCATCATCCTCGCTA | (3′) |
| | GG | |
| Primer 2 | GGATATC CTC GAG | Carboxyl Primer |
| SEQ ID NO: 6 | GAAGATAGCAAAGAAACGG | (5′) |
| | AAA | |

The gene was generated by PCR, using the above primers and genomic DNA from S. epidermidis strain RP62A as template. The PCR reaction was run at: 94° C. for 5 minutes, then 30 cycles of 94° C. for 45 seconds, 56° C. for 45 seconds, 72° C. for 3 minutes, followed by 10 minutes at 72° C. and 4° C. hold. The PCR product was purified on a 0.8% agarose gel, cleaned up with Qiagen gel extraction kit, and cut with XhoI and BlpI for 5.5 hours at 37° C.

The cut fragment was phenol/chloroform extracted and EtOH precipitated to remove enzyme activity and concentrate the sample. The fragment was resuspended in EB buffer (10 mM Tris-HCl pH 8.5) and used to ligate to pET-16b cut with XhoI, BlpI. Ligation was done at a 6:1 molar ration (insert to vector) with the Roche rapid ligation kit. The ligation reaction was transformed into NovaBlue competent cells and carbamicillin (50 μg/ml) resistant clones were used to make minipreps. Miniprep plasmids were screened by restriction digestion. Two clones were chosen to be sequence verified and to transform into BLR(DE3) competent cells for expression verification.

Expression after IPTG induction was checked from 6 ml cultures of clonal isolates from BLR(DE3) transformation plates. Samples were checked as uninduced, induced, induced soluble fraction and induced insoluble fraction. Expression was found to be very high but all of the product was in the insoluble fraction.

### SEQ ID NO: 2 Purification

Frozen recombinant E. coli cell paste (14 grams) was thawed and resuspended in two volumes of Lysis Buffer (50 mM sodium phosphate, pH 8.0, 0.15 M NaCl, 2 mM magnesium chloride, 10 mM imidazole, 0.1% Tween-80, Benzonase (EM #1.01697.0002) was added to the cell suspension at 250 Units/mL), and protease inhibitor cocktail was added to the cell suspension at one tablet per 50 ml (Complete™, EDTA-Free, Roche # 1873580). A lysate was prepared with a microfluidizer. The lysate was clarified by centrifugation at 10,000 xg for 45 minutes at 4° C. The supernatant was filtered through a 25 mm 0.2 micron Millipore Millex syringe filter. The Filtered Supernatant was added to Ni-NTA agarose chromatography resin (Qiagen #30250) and the slurry was mixed for approximately 16 hours at 4° C. The slurry of chromatography resin was poured into a chromatography column and the non-bound fraction was collected by gravity from the column outlet. The column was washed with ten column volumes of Wash Buffer (50 mM sodium phosphate, pH 8.0, 0.5 M NaCl, 2 mM magnesium chloride, 0.1% Tween-80, and 20 mM imidazole). The column was eluted with six column volumes of Elution Buffer (50 mM sodium phosphate, pH 8.0, 2 mM magnesium chloride, 0.1% Tween-80, and 0.3 M imidazole).

Fractions containing protein identified based on the SDS/PAGE and fractions containing the highest protein concentrations were pooled to make the Ni-IMAC Product. The Ni-IMAC Product was fractionated by SEC. SEC fractions containing the product protein were identified by SDS/PAGE with Coomassie staining. Product-containing SEC fractions were pooled to make the SEC Product. The filtrate was sterile-filtered and adsorbed on aluminum hydroxyphosphate adjuvant at a final concentration of 0.2 mg/ml.

### Example 2

### Rat Indwelling Catheter Model (Multiple Immunizations)

SEQ ID NO: 2 and a rat indwelling catheter model was used to assess whether active immunization using SEQ ID NO: 1 related polypeptides can inhibit staphylococcal infection of implanted devices. A vaccine containing SEQ ID NO: 2 was obtained as described in Example 1.

Rats were purchased at 3-4 wks and immunized on day 0, 14 and 21 either IP with immunogen on aluminium hydroxide phosphate (“AHP”) (Klein et al., Journal of Pharmaceutical Sciences 89:311-321, 2000), or mock immunized with adjuvant alone. On day 35 the animals had surgery to place an indwelling catheter in the jugular vein. The animals were rested for approximately 10 days after surgery, at which time a sub-lethal challenge of S. epidermidis strain RP62A was given IV (5-7×10⁹ CFU). The rats were sacrificed 24 hours post challenge, and the catheters removed.

The presence of bacteria on the catheters was assessed by culturing the entire catheter on mannitol salt agar plates. If any sign of outgrowth was observed on the plate the catheter was scored as culture positive (Table 2). Sham immunized animals have>80% of the catheters colonized. For an immunogen to be considered protective<50% of the catheters are colonized by the challenge strain.

TABLE 2
Active Immunization Experiments using a Rat Indwelling Catheter Model

| | # Infected | |
|---|---|---|
| Vaccine | Catheters (24 hr) | % Infected Catheters (24 hr) |
| AHP Control | 19/20 | 95 |
| SEQ ID NO: 2-AHP | 8/20 | 40 |

Other embodiments are within the following claims. While several embodiments have been shown and described, various modifications may be made without departing from the spirit and scope of the present invention.

1. A polypeptide immunogen comprising an amino acid sequence at least 90% identical to SEQ ID NO: 1, wherein said polypeptide provides protective immunity against S. epidermidis and the polypeptide does not have an amino acid sequence provided by SEQ ID NO: 3.

2. The polypeptide of claim 1, wherein said polypeptide consists of an amino acid sequence at least 94% identical to SEQ ID NO: 1.

3. The polypeptide of claim 2, wherein said polypeptide consists essentially of SEQ ID NO: 1.

4. The polypeptide of claim 3, wherein said polypeptide consists of an amino acid sequence of SEQ ID NO: 1 or methionine-SEQ ID NO: 1.

5. An immunogen comprising an amino acid sequence at least 90% identical to SEQ ID NO: 1 and one or more additional regions or moieties covalently joined to said amino acid sequence at the carboxyl terminus or amino terminus, wherein each region or moiety is independently selected from a region or moiety having at least one of the following properties: enhances the immune response, facilitates purification, or facilitates polypeptide stability.

6. A composition able to induce a protective immune response in a patient comprising an immunologically effective amount of the immunogen of claim 1 and a pharmaceutically acceptable carrier.

7. The composition of claim 6, wherein said composition further comprises an adjuvant.

8. A nucleic acid comprising a recombinant gene comprising a nucleotide sequence encoding the polypeptide of claim 1.

9. The nucleic acid of claim 8, wherein said nucleic acid is an expression vector.

10. A recombinant cell comprising the nucleic acid of claim 8.

11. A method of making a S. epidermidis polypeptide that provides protective immunity comprising the steps of:
(a) growing the recombinant cell of claim 10 under conditions wherein a polypeptide is expressed; and
(b) purifying said polypeptide.

12. A method of inducing a protective immune response in a patient comprising the step of administering to said patient an immunologically effective amount of immunogen comprising an amino acid sequence at least 90% identical to SEQ ID NO: 1.

13. The method of claim 12, wherein said patient is a human.

14. The method of claim 13, wherein said patient is treated prophylactically against S. epidermidis infection.

15. A method of inducing a protective immune response in a patient comprising the step of administering to said patient an immunologically effective amount of a polypeptide made by the method of claim 11.
